(19) United States
(12) Patent Application Publication
(10) Pub. No.: US 2016/0030632 A1
(43) Pub. Date: Feb. 4, 2016

(54) Bonding of Titanium Coating to Cast CoCr

(71) Applicant: PCC Structurals Inc., Portland, OR (US)

(72) Inventors: John Schleicher, Aloha, OR (US); James K. Barrett, Milwaukie, OR (US); James Ault, Portland, OR (US)

(21) Appl. No.: 14/771,877

(22) PCT Filed: Mar. 5, 2013
(86) PCT No.: PCT/US2013/029063

§ 371 Date: Sep. 1, 2015

(51) Int. Cl. A61L 27/30; A61L 27/04; A61F 2/00; B32B 15/01; C23C 4/08; C23C 4/18

(52) U.S. Cl. CPC A61L 27/306; C23C 4/08; C23C 4/18; A61F 2/0077; B32B 15/01; A61L 27/045; A61F 2002/0081; A61F 2002/0086; A61L 2430/02; A61L 2420/02

(57) ABSTRACT

Described is a medical implant having a CoCr cast body and a commercially pure (CP) Ti coating. The CP Ti coating is diffusion bonded to the CoCr body and is 5-3000 μm thick. Also described is a process for producing the medical implant that includes preparing the cast CoCr body for coating, applying a coating using a cold spray process, and diffusion bonding the coating to the body using hot isostatic pressing.

### BACKGROUND OF THE INVENTION

1. Field of the Invention

The present invention is generally directed to cast CoCr medical implants coated with titanium and the process for coating such implants.

2. Description of Related Art

There are two categories of major joint orthopedic implants available on the market today, coated and non-coated. A non-coated product has no coating and typically relies on an adhesive such as bone cement to hold the joint in place. Coated products typically have a porous surface on the bone interface which has been applied by sintering or plasma/vapor deposition. The presence of the porous surface is believed to allow for better bone in-growth of the orthopedic implant as well as improved corrosion resistance and fatigue life.

For example, it is known to surface treat medical implants made of Ti-, Co- and Fe-based alloys. The surface may be mechanically treated to increase roughness for better bone in-growth or coated with a porous coating material. A common method of applying such coatings is by chemical vapor deposition (CVD) of the coating material on a metallic substrate surface. Alternatively, coatings can be applied using a plasma spray process. Sintering is traditionally also used after application of the coating material in order to cause diffusion bonding of the porous coating to the metallic substrate surface. In another known process, a coating can be applied by applying porous beads to the surface by hand, through a fluidized bed, or using a rainfall-type apparatus.

These known processes for applying a coating to a medical implant have some significant disadvantages. For one, commercially pure (CP) titanium cannot be applied using CVD or plasma spray due to oxidation of the powder during the application. Moreover, current sintering techniques only allow bonding of similar metals, such as Ti to Ti or CoCr to CoCr, because a eutectic reaction can occur when sintering dissimilar metals. In addition, known processes can be very labor intensive and can produce inconsistent results. This is especially the case with coating processes involving application of porous beads by hand, using a fluidized bed, or rainfall-type apparatus. Coatings prepared by CVD also have a tendency to be thin and have low bond strength.

CP titanium is known to provide better bone in-growth than CoCr or Fe based coatings because of its better biocompatibility. CoCr alloys, such as those meeting the ASTM F-75 standard, are known to provide good wear resistance for medical implants. Thus, an object of the present invention is to develop CP Ti-coated Co- or Fe-based alloy medical implants and processes for their manufacture.

### SUMMARY OF THE INVENTION

In one non-limiting embodiment, the invention provides a medical implant comprising a cast or forged CoCr alloy body with a commercially pure Ti coating on the surface thereof.

In another non-limiting embodiment, the invention provides a process for coating a CoCr alloy medical implant comprising applying a coating of commercially pure Ti to the surface of the medical implant using a cold spray process and diffusion bonding the coating and the CoCr alloy medical implant using hot isostatic pressing.

### BRIEF DESCRIPTION OF THE DRAWINGS

FIG. 1 is a schematic of an embodiment of a cold spray system according to the present invention.

FIGS. 2A and 2B are, respectively, a process flow diagram of the process flow of a prior art process of preparing a porous-coated medical implant and a process flow diagram of a process according to the present invention.

FIG. 3 is a photograph of a coated plate prepared according to the process of the present invention.

FIG. 4 is a photograph of a series of test slugs from the coated plate of FIG. 3.

FIG. 5 is a series of photographs of a series of sectioned coated plates prepared according to the process of the present invention.

FIG. 6 is a series of photographs of the coated plates of FIG. 5 showing the test slugs thereof.

FIGS. 7-19 are photographs and spectra analyses obtained through analysis of a series of samples of coated plates prepared according to the process of the present invention as described below.

### DETAILED DESCRIPTION

The present invention is directed to a cast or forged medical implant of a Co- or Fe-based alloy, and preferably an CoCr alloy, such as an alloy meeting the ASTM F-75 standard (Co-28Cr-6Mo), that has been coated with commercially pure (CP) Ti as well as processes of manufacturing such coated implants. CP Ti is generally understood in the art as being unalloyed, ASTM Grade 1, 2, 3 or 4 titanium. The CP Ti coating is diffusion bonded to the CoCr substrate.

A cast or forged CoCr alloy medical implant, such as a knee, hip or stent, can be prepared for coating with CP Ti by mechanical or chemical cleaning. A 5-3000 μm thick, such as a 500 to 1500 μm thick, CP Ti coating is then applied using cold spray technology. Cold spray technology uses a supersonic gas jet to propel small particles of a coating metal towards a substrate. Upon impact with the substrate, the coating particles thermomechanically bond to the substrate. Cold spray technology has been discovered to allow a CP Ti coating to be applied to a CoCr substrate while avoiding many of the problems associated with CVD and plasma coating, such as oxidation of the coating and incompatibility of the coating and substrate materials.

The substrate may be a CoCr alloy medical implant that has been cast or forged. Particles of CP Ti having a diameter of between 1 and 100 μm, such as between 20 and 50 μm, are directed at the CoCr substrate surface as a particle stream through a nozzle using pressurized inert gas. The pressurized inert gas is typically supplied at a pressure of between 1 and 10 MPa, such as 2.5 to 6.5 MPa. The temperature of the pressurized inert gas is typically between 0 and 1300° F., such as between 150° F. and 1000° F. This temperature can be adjusted using a heater component disposed within the system.

Sufficient CP Ti particles should be directed to the substrate surface to achieve a coating thickness of between 5 and 3000 μm, such as between 500 and 1500 μm. The amount of CP Ti particles can be adjusted by adjusting the concentration of the particles within the particle stream, or by prolonging the duration of the spray process. The invention is not intended to be limited by the thickness of the coating, and the techniques described herein are applicable to achieve a wide range of possible coating thicknesses.

The velocity of the particles in the particle stream is typically maintained between 300 and 1500 m/sec, such as between 500 and 1000 m/sec. In most operations, the gas used in the supersonic gas jet is an inert gas, such as nitrogen or helium. However, in processes using gas near the lower end of the pressure and velocity range, compressed air may be used.

After coating the CoCr medical implant with CP Ti, the implant can be transferred to a hot isostatic press. Hot isostatic pressing (HIP) can be performed at a temperature between 900 and 1850° F., such as between 1600 and 1800° F., and a pressure between 10,000 and 25,000 psi, such as between 14,000 and 16,000 psi, for 1 to 5 hours, and typically for more than 2 hours. In a particularly preferred embodiment, HIP is performed at between 1650 and 1750° F. and 14,500 psi for at least 120 minutes. HIP causes the diffusion of the substrate materials, such as Co, Cr, and Mb, into the CP Ti of the coating, thereby strengthening the adhesion of the coating to the substrate. Care should be maintained that the HIP temperature is not set so high as to cause a eutectic condition between the Co and Ti. Following HIP, the medical implant can then be cooled to room temperature, machined, and polished according to known techniques.

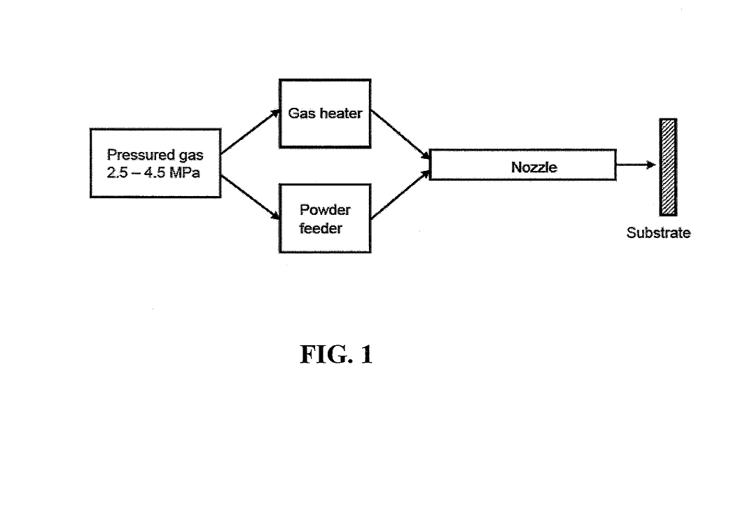

FIG. 1 illustrates a preferred embodiment of the cold spray system according to the present invention. The system can include a pressurized gas source, a gas heater, a coating powder feeder and a nozzle. In this embodiment, gas from the pressurized gas source is fed to the gas heater, where it is heated. Typical gases include air, nitrogen, helium or a mixture thereof. Alternatively, the pressurized gas can be heated to the required temperature within the pressurized gas source, after mixing with the coating powder, within the nozzle, or at another location within the system. While not intended to be limited, the pressurized gas should be heated to a temperature sufficient to ensure that the particle stream that is directed to the substrate surface is between 0 and 1300° F., preferably between 150 and 1000° F., and more preferably between 500 and 1000° F. The heater is preferably an electric heater, such as those commercially available in the field. Pressurized gas from the gas source can also be fed to the coating powder feeder, where CP Ti metal powder is mixed into the gas stream. Alternatively, the coating powder feeder can have its own pressurized gas source associated therewith. Typical powder feed rates are between about 10 and about 30 lbs/hr and the combined flow rate of the pressurized gas source to the heater and the powder feeder should typically be about 30 to 100 ft³/min.

The pressurized gas stream containing the CP Ti powder can then be fed to the nozzle. The nozzle is used to focus the gas stream containing the CP Ti powder particles and direct it toward the substrate surface in the form of a stream, or spray, of particles traveling at supersonic speeds. The particle velocity should typically be within the range of 300 to 1500 m/sec. Upon impacting the substrate surface, the particles are deposited by means of ballistic impingement to form a coating. Formation of the coating through this method involves mechanical mixing of the particles of the coating with the substrate material at the interface.

The systems and processes described herein can also be automated by providing communication means, such as in the form of wired and/or wireless data communication links, between the various components of the system and one or more control units, each of which may be a computer. Automation typically allows for automatic control of the powder feed rate, velocity of the particle stream, gas flow rate, and spray distance. Such control can be based on parameters that are set by the operator as well as feedback learned by the control unit from monitoring different components of the system. Using such inputs, the control unit can determine and adjust the different process parameters accordingly to achieve optimal results.

The use of a cold spray process according to the present invention achieves several advantages over the prior art coating methods. For one, the cold spray process described herein allows for oxide free coatings to be formed. In addition, the cold spray process enables bonding of dissimilar materials, most notably bonding of CP Ti with a Co-based substrate such as CoCr, which is not possible with existing processes which employ sintering techniques. Indeed, the cold spray process of the present invention can eliminate the need for sintering of the coated material at all.

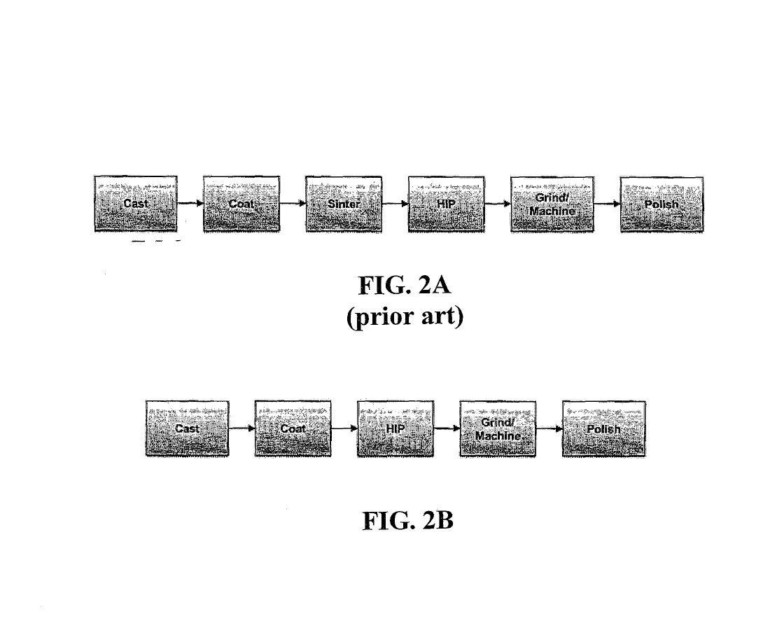

FIGS. 2A and 2B represent a comparison of the process flow steps in a prior art process for forming a porous-coated medical implant with the process flow steps in one embodiment of the cold spray process of the present invention. As can be seen from FIG. 2, the cold spray process can eliminate the need for post-coating sintering of the coated substrate, among other advantages as described herein.

While primarily focused on the formation of a coating, it should be understood that this process may be useful in other applications, such as the formation of a bulk structure.

The benefits of the present invention will be further apparent by reference to the following examples.

### EXAMPLES

Two processes for applying a coating according to the present invention were evaluated. The first was a cold spray high pressure process, and the second was a cold spray low pressure process.

### High Pressure Process

A sample CoCr cast plate meeting ASTM F-75 was prepared by PCC (92807-00001; metal lot 70353). The sample was coated with CP Ti using a high pressure process according to the invention. The chemical composition of the CP Ti powder used is presented in Table 1 below:

TABLE 1
CP Ti chemical analysis (in %)

| | | | | | | | |
|---|---|---|---|---|---|---|---|
| Ag | Al | B | Ba | Bi | Ca | Cl | Ce |
| | 0.022 | <0.0005 | | <0.02 | 0.0007 | <0.01 | |
| Co | Cr | Cu | Fe | Ga | Ge | Hf | In |
| <0.01 | <0.006 | <0.005 | 0.042 | | | <0.01 | |
| K | La | Mg | Mn | Mo | Na | Nb | Nd |
| | | <0.01 | <0.005 | <0.005 | 0.0005 | <0.01 | |
| Ni | P | Pd | Re | Sb | Sc | Se | Si |
| 0.011 | | <0.005 | | | | | 0.0059 |
| Sn | Sr | Ta | Ti | Te | V | W | Y |
| <0.01 | | <0.005 | 99.8 min | | <0.005 | <0.005 | <0.0005 |
| Zn | Zr | C | F | H | O | N | S |
| | <0.005 | | | 0.075 | 0.39 | 0.023 | |
| H Loss | H2O | Insoluble | Appearance | LOI | LOD | Volatile | Other Max |

The CP Ti coating was applied by using pressurized gas. The thickness variation of the coating was observed to be between 1 and 3 mm.

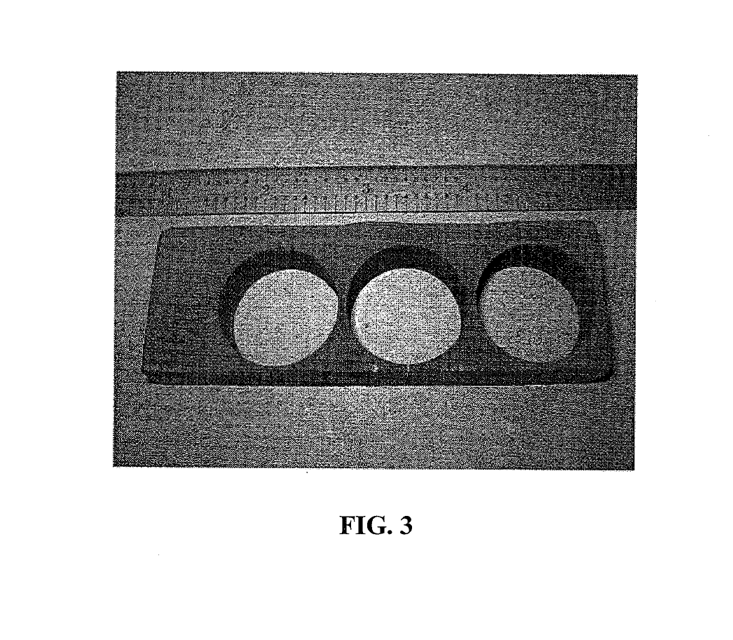

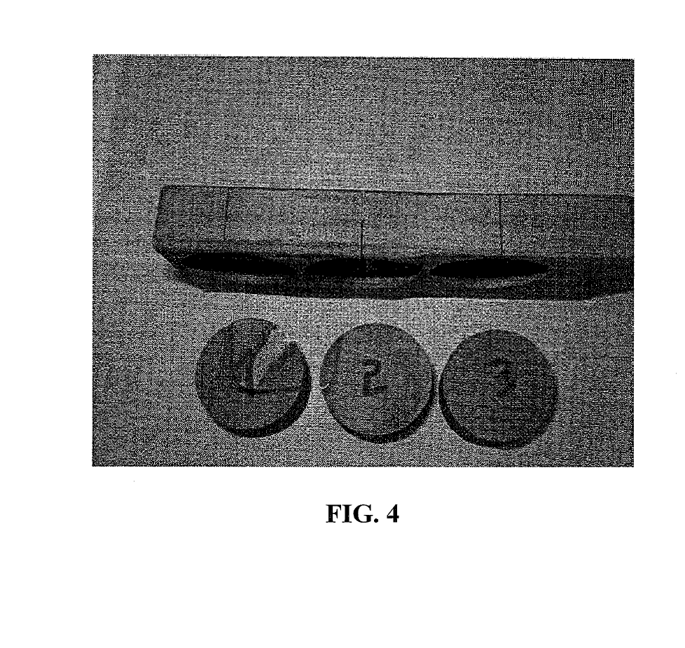

After coating, the sample plate was cut in half using electric discharge machining (EDM). From each half, three test slugs were removed using EDM. Adhesion and metallographic properties of the first half of the plate and the associated test slugs were evaluated without first subjecting the sample to hot isostatic pressing (HIP). The second half of the test plate and the associated test slugs underwent HIP and then were evaluated for adhesion and metallographic properties to determine the effect of HIP on these properties. FIG. 3 shows one half of the coated surface test plate with the three test slugs removed and FIG. 4 shows the coated surface of a test plate along with the three test slugs.

The three test slugs from the first half of the test plate were tested for adhesion properties as coated (but without HIP) according to ASTM F1147. The results for each test slug and failure mode, which for this test occurred by pulling the coating away from the substrate, are presented below in Table 2:

TABLE 2
Adhesion test results (high pressure process, no HIP)

| Test Slug | Bond strength (psi) | Failure mode |
|---|---|---|
| 1 | 6,324 | Coating to substrate |
| 2 | 8,322 | Coating to substrate |
| 3 | 6,405 | Coating to substrate |

A metallographic evaluation was also performed (Lisin Job #332-10-201) on the remaining section of the first half of the test plate (i.e. the portion remaining after removal of the test slugs). The evaluation included analyzing the micros at 50× and at 100×, the depth of the coating, the porosity percentage, and the porosity size. A scanning electron microscope (SEM) analysis was also performed, including a line scan from the coating surface to a depth 3 mm below at 0.5 mm increments along with an oxygen analysis on the coating and substrate. The findings of the metallographic evaluation include: the coating was easily removed by destructive means; no diffusion was detected; and the coating was dense and exhibited minimal porosity.

The second half of the test plate was subjected to HIP at 1750° F. and 14,750 psi for 120 minutes. The plate was then subjected to natural cool to the unload temperature, which was less than 400° F. After completion of HIP, three test slugs were removed from the test plate using EDM.

The three test slugs were then subjected to an adhesion test according to ASTM F1147. The results of the adhesion test and failure mode for each test slug are presented below in Table 3:

TABLE 3
Adhesion test results (high pressure process, after HIP)

| Test Slug | Bond strength (psi) | Failure mode |
|---|---|---|
| 1 | 7,245 | Button to mandrel |
| 2 | 7,088 | Button to mandrel |
| 3 | 7,332 | Button to mandrel |

A metallographic analysis was also performed (Lisin Job #332-10-202) on the remaining section of the second half of the test plate. The evaluation included analyzing the micros at 50× and at 100×, the depth of the coating, the porosity percentage, and the porosity size. A SEM analysis was also performed, including a line scan from the coating surface to a depth 3 mm below at 0.5 mm increments along with an oxygen analysis on the coating and substrate. The findings of the metallographic evaluation include: the coating was difficult to remove by destructive means; diffusion was detected; and the coating was dense and exhibited minimal porosity.

The results of the tests show that the use of HIP increased the bond strength of the coating, made the coating more difficult to remove and caused diffusion bonding of the coating.

### Low Pressure Process

Sample CoCr cast plates meeting ASTM F-75 were prepared by PCC (92807-00001; metal lot 70353). The samples were coated with CP Ti using a low pressure cold spray process using the same CP Ti metal powder as discussed above in the high pressure process.

The thickness variation of the coating was observed to be between 1 and 3 mm. After coating, the plates were cut in half using EDM. The first half of each plate was held for future trials. The second half of each plate was then subjected to HIP at 1750° F. and 14,750 psi for 120 minutes. The plate was then subjected to natural cool to the unload temperature, which was less than 400° F. It was observed that, without HIP, flaking occurred when attempting to produce test slugs. Therefore, evaluation of a low pressure cold spray coated, but not HIP treated, sample was not completed.

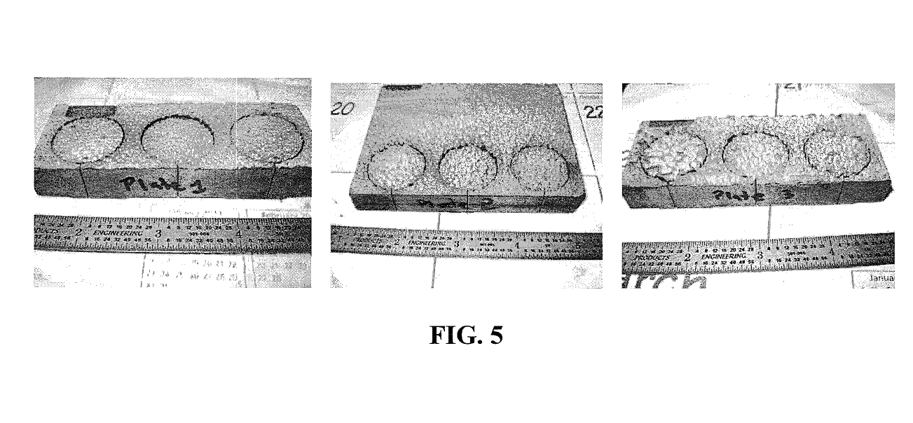

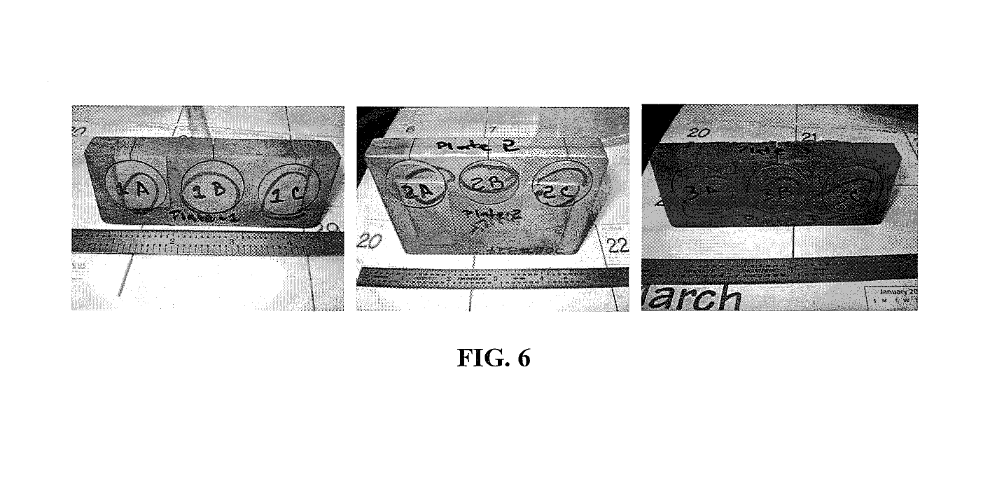

Following HIP of the plate halves, three test slugs were prepared from each plate half using EDM. FIG. 5 shows the coated surface of a plate as sectioned. FIG. 6 shows different test plates with the test slugs.

Each of the test slugs was tested for adhesion properties according to ASTM F1147. The results for each test slug and failure mode are presented below in Table 4:

TABLE 4
Adhesion test results (low pressure process, after HIP)

| Test | | | |
|---|---|---|---|
| Plate | Test Slug | Bond Strength (psi) | Failure Mode |
| 1 | A | 7,371 | 100% adhesive to coating |
| 1 | B | 6,929 | 100% adhesive to coating |
| 1 | C | 5,353 | 60% adhesive to coating, 40% |
| | | | coating to coating |
| 2 | A | 5,958 | 30% adhesive to coating, 70% |
| | | | coating to coating |
| 2 | B | 6,258 | 85% adhesive to coating, 15% |
| | | | coating to coating |
| 2 | C | 7,854 | 100% adhesive to coating |
| 3 | A | 7,148 | 100% adhesive to coating |
| 3 | B | 6,478 | 100% adhesive to coating |
| 3 | C | 7,662 | 100% adhesive to coating |

A metallographic evaluation was also performed (Lisin Job #332-11-223; Exova Job #126682) on the remaining section of the second half of the test plates, including a scanning electron microscopy and energy dispersive x-ray analysis of the coated surface and of metallographic sections through the coated surface to characterize the coating and substrate. The findings of the metallographic evaluation include: The coatings were substantially more adherent than previously examined samples. Several hard blows to a sharp chisel with a two pound hammer were required to dislodge the coating. The coating exhibited a non-uniform porous structure. In general, the coating was more dense at mid thickness locations and toward the substrate. Porosity ranging from approximately 25% to approximately 39% was apparent near the exposed surface of the coating, within the industry standard of 20-75%. The coating included essentially pure titanium toward the exposed surface. A thin oxide film was present on the exposed surface. Diffusion of cobalt into the coating was measured to a maximum depth of approximately 0.0087 inch. Chromium did not appear to diffuse from the substrate into the coating. Significant diffusion of titanium into the cobalt alloy substrate was not detected at a depth of approximately 0.0035 inch from the interface. The porosity of the plates prepared according to the low pressure process exceeded the FDA requirements for porosity in a coating for medical implants.

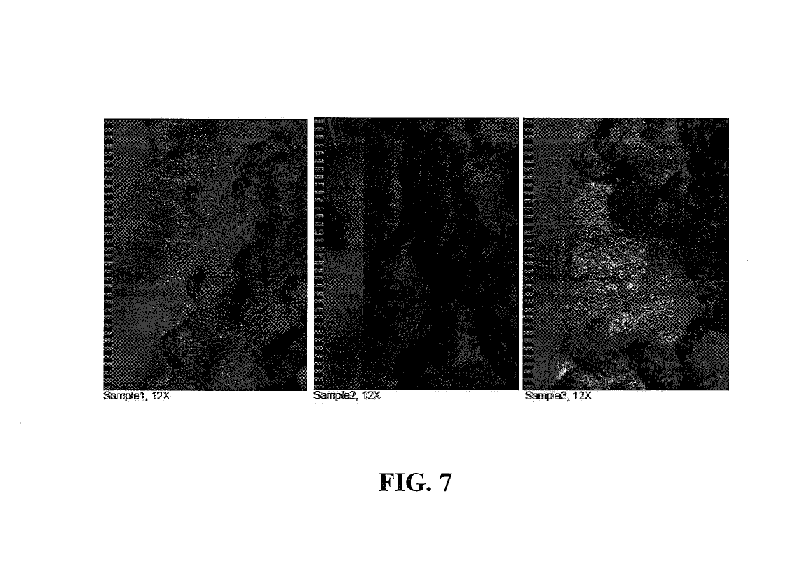

FIG. 7 represents higher magnification views of cuts through the porous coated surfaces of samples from Test Plates 1-3. The porous coated surfaces consisted of steep or abrupt peaks and adjacent pits. The pattern appeared increasingly coarse between the samples. The coloring of the porous coated surface suggests that a thin oxide or nitride film may have been present on the surface of the porous titanium layer.

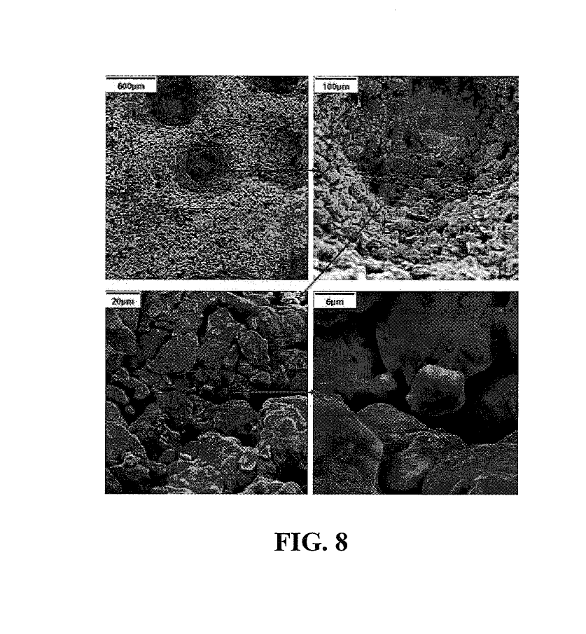

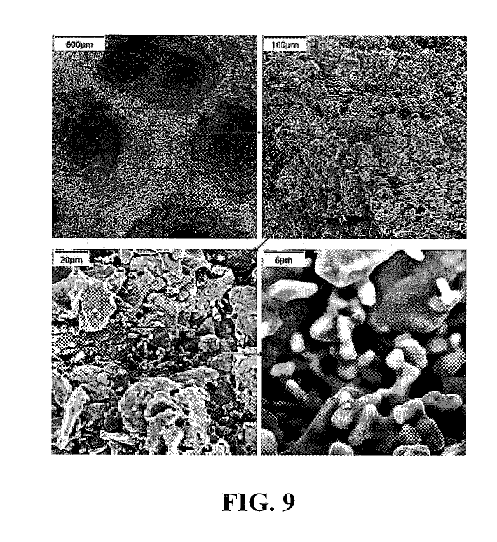

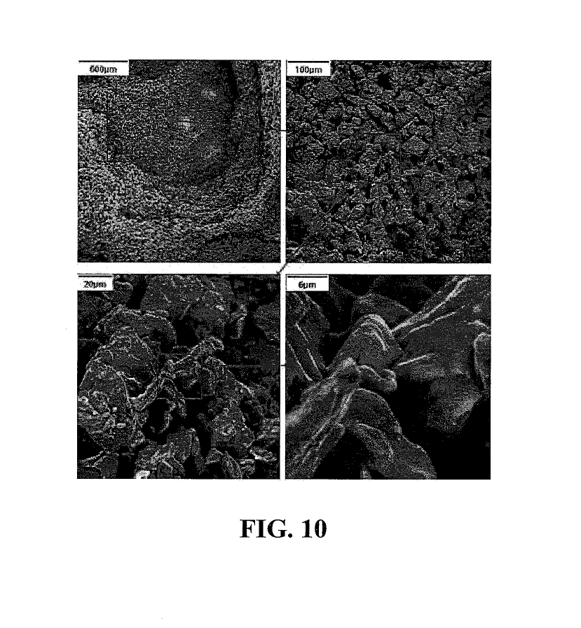

FIGS. 8-10 represent backscattered electron images acquired from a series of increasing magnification images of the coated surfaces of samples from Test Plates 1-3, respectively. Substantial surface connected porosity is apparent in each.

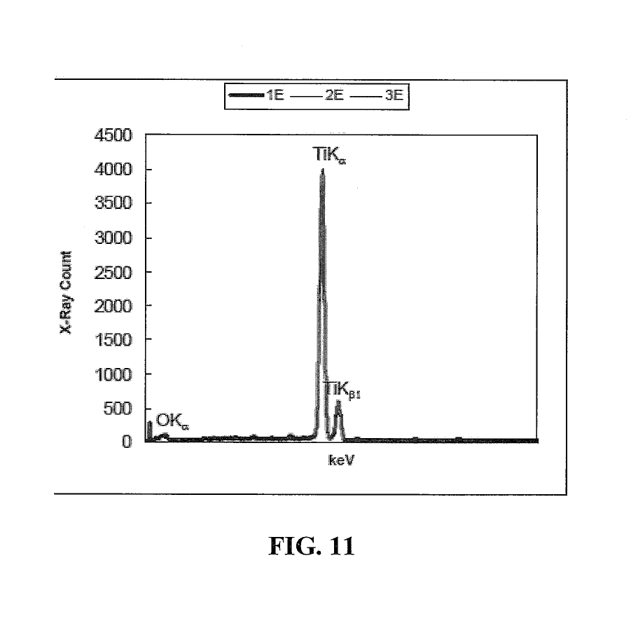

FIG. 11 represents an energy dispersive x-ray spectra acquired from the exposed coating surfaces of samples from Test Plates 1-3. Only titanium and trace amounts of oxygen were detected.

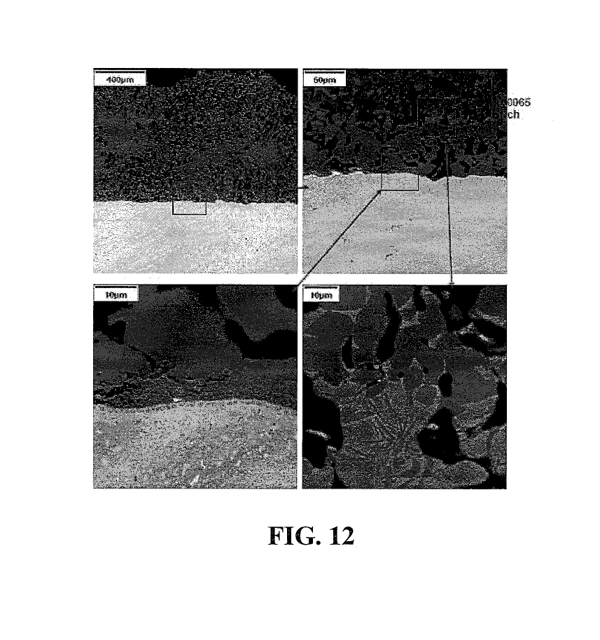

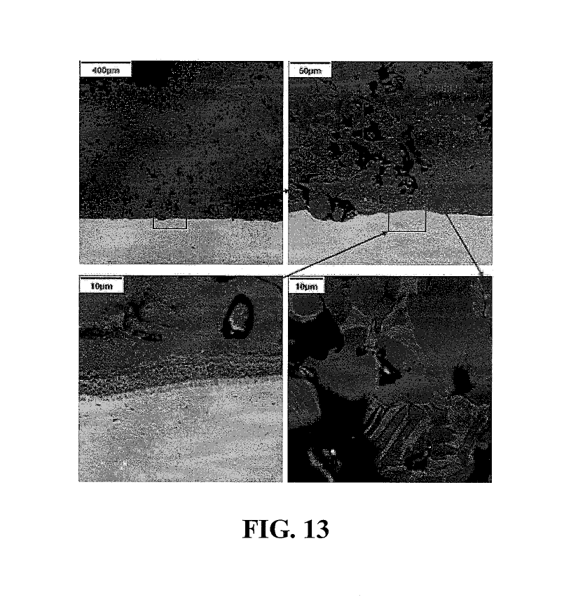

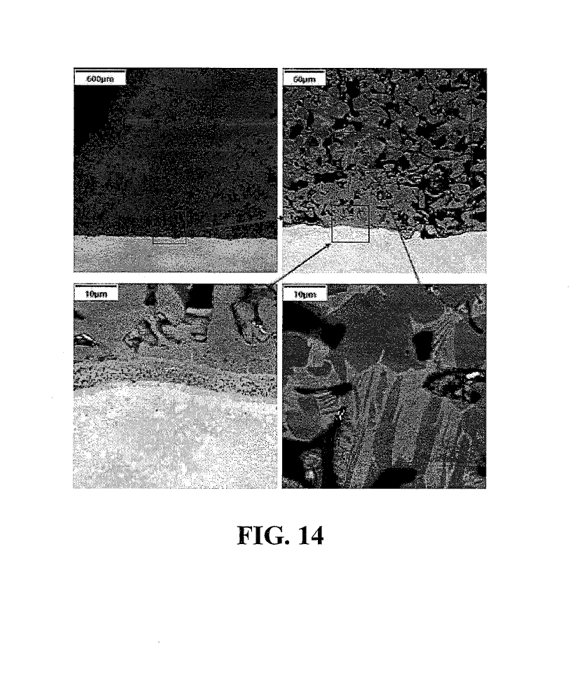

FIGS. 12-14 represent backscattered electron images acquired from a metallographic section through the interface area of samples from Test Plates 1-3, respectively. Diffusion of cobalt into the titanium coating is apparent as the lighter phase of the titanium porous coating. The cobalt enriched titanium appears to form a discrete phase rather than a continuously decreasing diffusion gradient. In Test Plate 1 (FIG. 12), the cobalt containing phase extended to a distance of at least 0.0065 inch from the interface; in Test Plate 2 (FIG. 13), the cobalt containing phase extended to a distance of at least 0.0087 inch from the interface; and in Test Plate 3 (FIG. 14) the cobalt containing phase extended to a distance of at least 0.0085 inch from the interface.

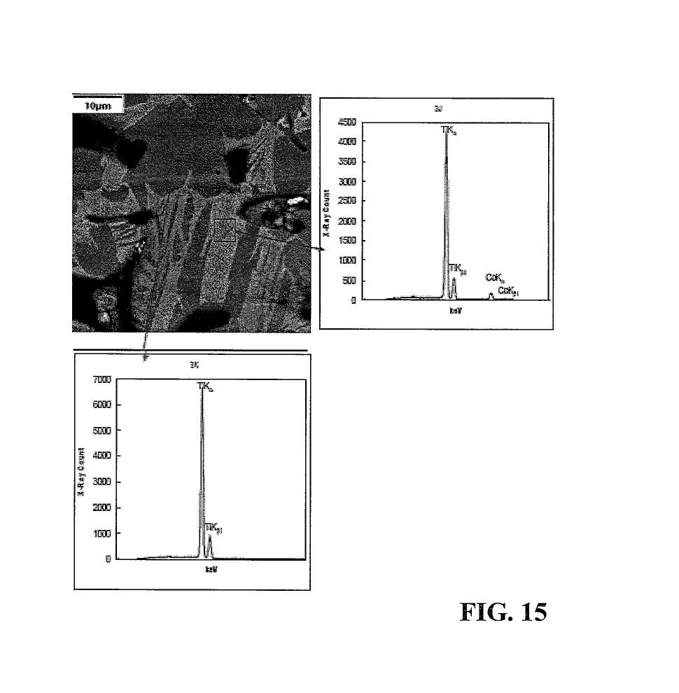

FIG. 15 represents backscattered electron images and energy dispersive x-ray spectra acquired from the high and low density phases on the titanium side of the interface of a sample from Test Plate 3. Cobalt appears to be confined to the high density phase. Chromium does not appear to have diffused with the cobalt.

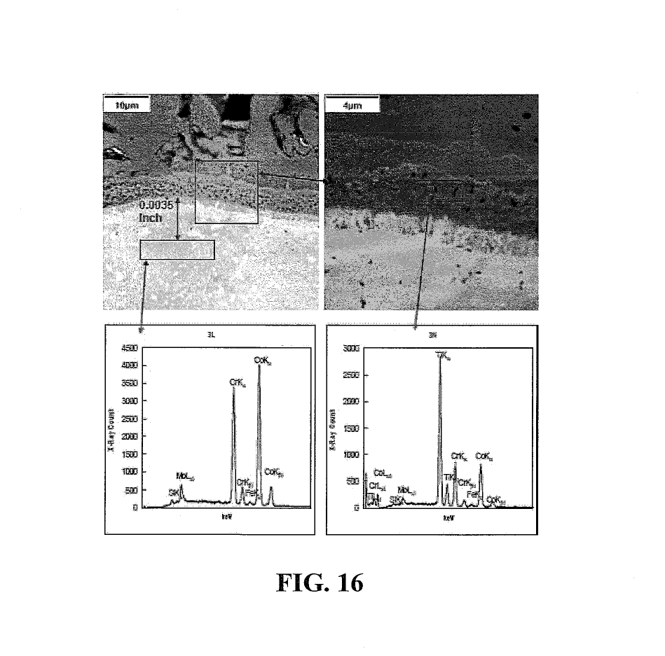

FIG. 16 represents backscattered electron images and energy dispersive x-ray spectra acquired from a metallographic section through the interface area of a sample from Test Plate 3. The interface appears to consist of four discrete layers. No titanium diffusion into the cobalt was detected at a depth of approximately 0.0035 inch from the interface.

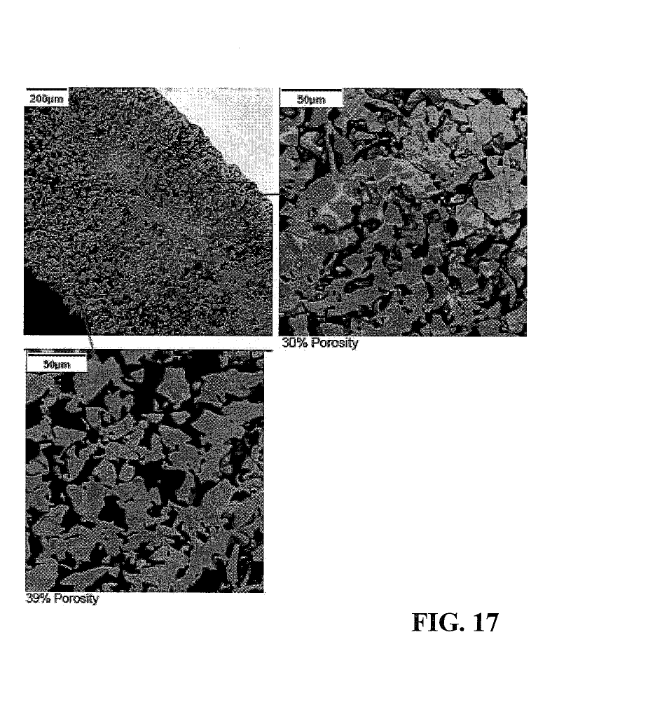

FIG. 17 represents backscattered electron images acquired from a metallographic section through a sample of Test Plate 1. Porosity is not uniform through the section. A denser region is apparent at an approximate mid-thickness location. Automated image analysis using Image J software indicates porosity area fractions of approximately 30% and 39% for the locations shown.

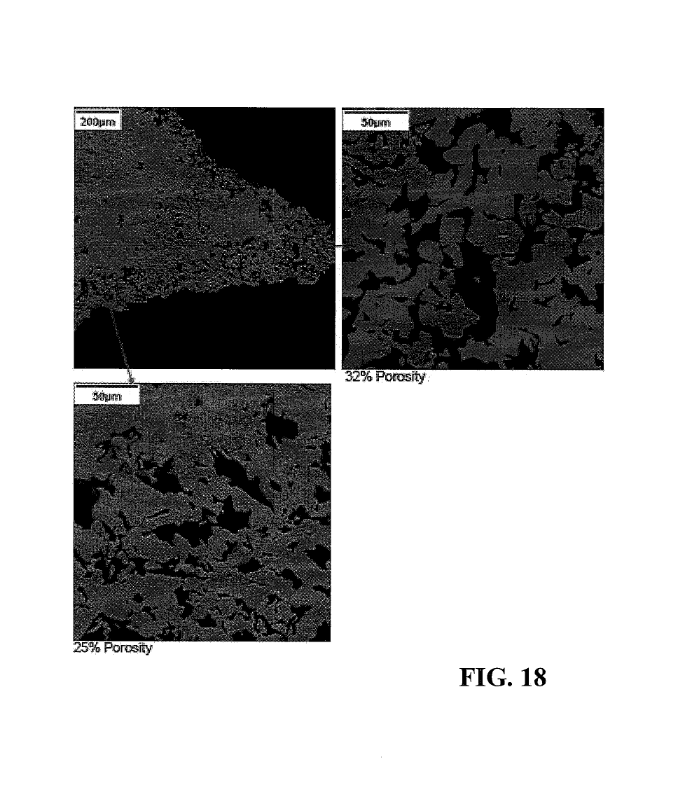

FIG. 18 represents backscattered electron images acquired from a metallographic section through a sample of Test Plate 2. Porosity is not uniform through the section. A less dense layer is apparent toward the outer surface. Automated image analysis using Image J software indicates porosity area fractions of approximately 32% and 25% for the locations shown.

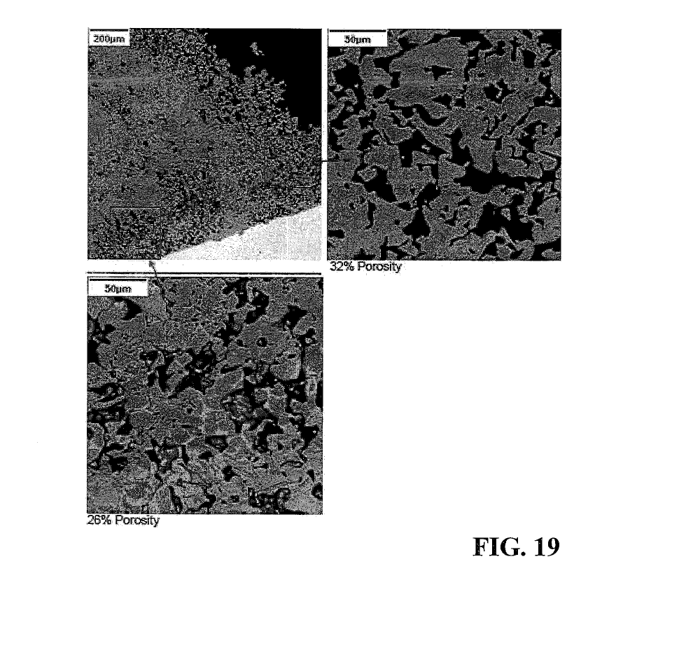

FIG. 19 represents backscattered electron images acquired from a metallographic section through a sample of Test Plate 3. Porosity is not uniform through the section. A less dense layer is apparent toward the outer surface. Automated image analysis using Image J software indicates porosity area fractions of approximately 32% and 26% for the locations shown.

From the data above, it was observed that the low pressure cold spray process produced a coating with higher porosity than the high pressure cold spray process.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. For example, it is to be understood that the present invention contemplates that, to the extent possible, one or more features of any embodiment can be combined with one or more features of any other embodiment.

The invention claimed is:

1. A process for coating a CoCr alloy medical implant comprising:
applying a coating of commercially pure Ti to the surface of the medical implant using a cold spray process; and
diffusion bonding the coating and the CoCr alloy medical implant using hot isostatic pressing.

2. The process according to claim 1, wherein the cold spray process comprises applying to the surface of the medical implant a particle stream comprising particles of commercially pure Ti and a pressurized gas.

3. The process according to claim 2, wherein the pressurized gas is supplied at a pressure of 1.0-10.0 MPa.

4. The process of claim 2, wherein the pressurized gas is an inert gas.

5. The process of claim 2, wherein the pressured gas is compressed air.

6. The process of claim 2, wherein the particle stream is at a temperature of 0-1300° F.

7. The process according to claim 2, wherein the particles of commercially pure Ti in the particle stream have a diameter of 1-100 μm.

8. The process of claim 2, wherein the particles of commercially pure Ti in the particle stream travel at a velocity of 300-1500 m/s.

9. The process of claim 1, wherein the hot isostatic pressing is performed at a temperature of 900-1850° F.

10. The process of claim 1, wherein the hot isostatic pressing is performed at a pressure of 10,000-25,000 psi.

11. The process of claim 1, wherein the hot isostatic pressing is performed at a temperature of 900-1850° F., a pressure of 10,000-25,000 psi, and for a time period of 1-5 hours.

12. The process of claim 2, wherein the hot isostatic pressing is performed at a temperature of 900-1850° F., a pressure of 10,000-25,000 psi, and for a time period of 1-5 hours.

13. A medical implant comprising a cast or forged CoCr alloy body with a commercially pure Ti coating on the surface thereof.

14. The medical implant according to claim 13, wherein the coating is 5-3000 μm thick.

15. The medical implant according to claim 14, wherein the coating is 500-1500 μm thick.

16. A coated CoCr alloy medical implant, wherein the coating is formed according to the process of claim 1.

17. A coated CoCr alloy medical implant, wherein the coating is formed according to the process of claim 12.
